(19) United States
(12) Patent Application Publication
(10) Pub. No.: US 2018/0099159 A1
(43) Pub. Date: Apr. 12, 2018

(54) HADRON THERAPY APPARATUS FOR ADAPTIVE TREATMENT IN NON-SUPINE POSITION

(71) Applicant: Ion Beam Applications S.A., Louvain-la-Neuve (BE)

(72) Inventors: Eric FORTON, Nil-Pierreux (BE); Damien BERTRAND, Dhuy (BE); Yves CLAEREBOUDT, Nil-Saint-Vincent (BE); Erik VAN DER KRAAIJ, Rixensart (BE)

(73) Assignee: Ion Beam Applications S.A.

(21) Appl. No.: 15/727,503

(22) Filed: Oct. 6, 2017

(30) Foreign Application Priority Data
Oct. 7, 2016 (EP) 16192770.2

(51) Int. Cl. A61N 5/10; A61B 6/03

(52) U.S. Cl. CPC A61N 5/1069; A61N 5/1049; A61B 6/0421; A61N 2005/1087; A61B 6/03

(57) ABSTRACT

The present disclosure relates to an apparatus including a hadron therapy device and a magnetic resonance imaging device (MRI). The MRI may be an open MRI for acquiring magnetic resonance data in an MRI imaging volume. A nozzle of the apparatus may be fixed and positioned for directing a beam along a beam path substantially along an axis or substantially perpendicularly to the axis. The apparatus may further include a patient support system adapted for supporting a patient in a non-supine position in the MRI. The present disclosure also relates to methods for adapting a treatment plan to movements of organs resulting from displacement of a patient from a supine position in which treatment plan imaging was performed to a non-supine position in which a treatment will be performed.

### CROSS-REFERENCE TO RELATED APPLICATIONS

The application claims the benefit of priority to European Application No. 16192770.2, filed Oct. 7, 2016, the contents of which are incorporated herein by reference.

### TECHNICAL FIELD

The present disclosure relates to a medical apparatus comprising a hadron therapy device and a magnetic resonance imaging device, and to a method for preparing a treatment plan for use in such a device.

### BACKGROUND

Hadron therapy (for example, proton therapy) for treating a patient may offer several advantages over conventional radiotherapy. These advantages are generally due to the physical nature of hadrons, including the fact that energy deposition curve in matter typically occurs along a curve presenting a sharp peak, the Bragg peak.

Hadron therapy usually requires the establishment of a treatment plan before any treatment can start. During this treatment plan, a 3D image, which may be a computer tomography scan (CT scan), an MRI image, and/or a PET scan, of the patient and target tissues may be performed. The 3D image may be used to characterize the target tissue and the surrounding tissues to be traversed by a treatment hadron beam for the treatment of a patient. The characterization may yield a 3D representation of the volume comprising the target tissue, and a treatment plan system may determine a range-dose calculated based on the nature of the tissues traversed by the hadron beam. The 3D image may be taken with a patient in supine position, i.e., lying horizontally with the face and torso facing up. In this position, the organs usually take a natural rest position. The treatment may be performed with the patient in the same supine position. This may minimize the risks that organs may have moved between the acquisition of the 3D image and the treatment.

The treatment plan may then be executed during a treatment phase including one or more treatment sessions during which doses of hadrons are deposited onto the target tissue. The position of the Bragg peak of a hadron beam with respect to the target spots of a target tissue, however, may suffer from a number of uncertainties including, for example:
the variations of the patient position, on the one hand, during a hadron therapy session and, on the other hand, between the establishment of the treatment plan and the hadron therapy session; and/or
the variations of the size and/or of the position of the target tissue and/or of the healthy tissues positioned upstream from the target tissue with respect to the hadron beam (the variation in position of the organs and target tissue may be emphasized when the patient is positioned during treatment in a non-supine position, e.g., seated or standing).

The uncertainty on the position of the patient and, in particular, of the target tissue may present challenges. Even with an accurate characterization by the 3D image, the actual position of a target tissue during a treatment session remains difficult to ascertain because, for example:
first, during an irradiation session, the position of a target tissue may change because of anatomical processes such as breathing, digestion, or heartbeats of the patient. Anatomical processes may also cause gases or fluids appearing or disappearing from the path of a hadron beam.
second, treatment plans are usually determined several days or weeks before a hadron treatment session starts and treatment of a patient may take several weeks distributed over several treatment sessions. During this time period, the patient may lose or gain weight, for example, therefore modifying, sometimes significantly, the volume of tissues such as fats and muscles.

The use of a magnetic resonance imaging device (MRI) coupled to a hadron therapy device may identify any variation of the size and/or the position of a target tissue. For example, U.S. Pat. No. 8,427,148 generally relates to a system comprising a hadron therapy device coupled to a MRI. Said system may acquire images of the patient during a hadron therapy session and may compare these images with CT scan images of the treatment plan. An example of a suitable MRI includes, but is not limited to, a device described in European Pat. No. 0186238.

Hadron therapy devices may comprise a plurality of treatment rooms. A typical installation, such as that described in U.S. Pat. No. 4,870,287, may comprise three treatment rooms having a gantry and one treatment room having a fixed beam. Gantry treatment rooms may allow irradiation of a patient from any direction, e.g., by positioning the gantry at a desired angle. In combination with a rotation of the patient support, this may allow full flexibility, i.e., irradiation of the patient from any direction in a full sphere (47). This flexibility may be useful to the therapist establishing the treatment plan, but may also represent a significant space and cost. A gantry is generally a large mechanical structure, supporting heavy magnets for guiding the beam. The structure of a gantry for proton therapy may have, for example, a length of 10 m, a diameter of 10 m and a weight of 100 tons. A gantry for carbon therapy may be much larger and heavier (e.g., length of 25 m, diameter of 13 m, and weight of 600 tons). The cost of the gantry, including the shielding enclosing said gantry, may represent up to or more than 60% of the cost of a treatment room.

Treatment rooms having one or more fixed beam lines may reduce this cost. An example of such a hadron therapy device is disclosed in PCT Pat. publication No. WO 03/092812. This device may comprise a plurality of fixed magnetic channels, in a vertical plane. A deflecting magnet may be provided at the end of each channel, for directing the beam from a wider range of directions.

Hadron therapy devices having a fixed beam line, more specifically a horizontal fixed beam line, are used, e.g., at the Harvard Cyclotron Laboratory. In this centre, treatments of patients have been performed, especially for intercranial and eye tumours. It may be convenient to treat such tumours with a fixed horizontal beam line, because such organs move less than other organs. However, there is a need for hadron therapy device having a fixed beam line for treating other tumours in the body.

### SUMMARY

Embodiments of the present disclosure may provide a medical apparatus comprising a hadron therapy device having a fixed beam line, adapted for treating a patient in a non-supine position.

In a first aspect, a medical apparatus may comprise:
a) a hadron therapy device comprising a hadron source having a nozzle adapted for directing a hadron treatment beam along a beam path to a target volume; and
b) a magnetic resonance imaging device (MRI).

In some embodiments,
the MRI may comprise two coils arranged at a distance and configured for generating a magnetic field in a direction along an axis in an MRI imaging volume between said two coils and around said axis, for acquiring magnetic resonance data in said MRI imaging volume, said MRI imaging volume encompassing said target volume, said imaging volume having a centre;
said nozzle may be fixed and positioned for directing a beam along a beam path substantially along said axis, the angle between said axis and said beam path may be smaller or equal to 20°, or substantially perpendicularly to said axis, the angle between a perpendicular to said axis and said beam path may be smaller or equal to 20°; and
the medical apparatus may further comprise a patient support system adapted for supporting a patient in a non-supine position.

In an embodiment, said patient support system may be adapted for supporting a patient in a seated position. The seated position may comprise positions with the patient leaning backwards or forwards.

In another embodiment, said patient support system may be adapted for supporting a patient in a standing position. The support may have a vertical part with means for immobilizing the patient, the patient having his back or his front in contact to the vertical part.

Said patient support system may be adapted for being rotated around a vertical axis.

In still another embodiment, said patient support system may be adapted for supporting a patient in a prone position. For example, the patient may be lying on a generally flat support or a support having a shape adapted to the morphology of the patient, depending on the needs of the treatment. In additional, the patient may be bound to the support by immobilization means.

Said patient support system may be adapted for being rotated around a horizontal axis, and said axis may be a longitudinal axis of said patient support.

In some embodiments, the apparatus may further comprise a controller adapted for instructing said apparatus to perform a method of the present disclosure.

In some embodiments, said nozzle may be positioned at a distance larger than 2 m, e.g., larger than 3 m, from said center of said MRI imaging volume. The distance may be measured from the point where the hadron beam exits the nozzle to the center of the MRI imaging volume.

In one embodiment, said nozzle may be positioned for directing a beam in a first direction along a beam path substantially along said axis, and the beam path may include a first pair of magnets S1x, S1y configured for steering the beam in two orthogonal directions both perpendicular to said first direction, and a second pair of steering magnets S2x, S2y configured for steering the beam in a direction parallel to said first direction.

In a second aspect, a method for preparing a treatment plan for treating a patient may comprise:
obtaining a 3D image of a target volume of said patient using at least one of the following: a CT-scan, an MRI (as described above), and/or a PET imaging system, said patient being in a supine position;
determining a treatment plan based on said 3D image;
determining a displacement of said target volume within said patient resulting from said patient being moved from said supine to said non-supine position;
adapting said treatment plan to said displacement; and
positioning said patient in said apparatus said patient being in said non-supine position.

Determining a displacement may comprise:
obtaining a first MRI image from said MRI, the patient being in said non-supine position;
obtaining a second MRI image from said MRI, the patient being in a supine position; and
determining said displacement of said target region from the comparison of said first and second MRI image.

Alternatively or concurrently, determining a displacement may comprise:
obtaining a first MRI image from said MRI, the patient being in said non-supine position; and
determining said displacement of said target region from the comparison of said first MRI image and said 3D image.

In a third aspect, a method for preparing a treatment plan for treating a patient may comprise:
obtaining a 3D image of a target region of said patient using at least one of the following: a CT-scan, an MRI (as described above), or a PET imaging system, said patient being in a non-supine position;
determining a treatment plan based on said 3D image; and
positioning said patient in said apparatus said patient being in said non-supine position.

In a fourth aspect, a method for treating a patient, wherein said treatment plan comprises a plurality of spots each having a spot position in said target region, may comprise:
1) preparing a treatment plan (as described above);
2) irradiating one or more spots of said plurality of spots
3) acquiring an image of said MRI imaging volume using said MRI;
4) comparing said image with said 3D image and adapting said treatment plan according to the differences; and
5) repeating steps 2) to 4) until all spots of said plurality have been irradiated.

As used herein, a nozzle that is fixed and positioned for directing a beam refers to a nozzle of what is generally named, as discussed above, a fixed beam line, i.e., a nozzle that cannot move. Typically, the nozzle of a fixed beam line may be attached to a support that is fixed to the floor level of the treatment room, in contrast to a nozzle mounted on a gantry structure, where the nozzle, together with a part of the beam line, may rotate around the rotation axis of the gantry.

As used herein, supine position refers to lying horizontally with the face and torso facing up, but also positions where the legs or knees or arms are displaced with respect to a supine torso. Similarly, standing and seated positions include positions where the patient is tilted forwards or backwards.

### SHORT DESCRIPTION OF THE DRAWINGS

These and further aspects of the present disclosure will be explained in greater detail by way of example and with reference to the accompanying drawings in which:

FIG. 1A represents schematically a side view of a medical device according to an example embodiment of the present disclosure, wherein the nozzle is positioned for directing a beam substantially in the direction of the axis of the MRI, the axis of the MRI being horizontal.

FIG. 1B represents schematically a top view of the example medical device of FIG. 1A.

FIG. 2 represents schematically a top view of a medical device according to an example embodiment of the present disclosure wherein the nozzle is positioned for directing a beam in a direction substantially perpendicular to the direction of the axis of the MRI, the axis of the MRI being horizontal.

FIG. 3 represents schematically a side view of a medical device according to an example embodiment of the present disclosure wherein the nozzle is positioned for directing a beam substantially in the direction of the axis z of the MRI, the axis of the MRI being vertical.

FIG. 4 represents schematically a side view of a medical device according to an example embodiment of the present disclosure wherein the nozzle is positioned for directing a beam in a direction substantially perpendicular to the direction of the axis of the MRI, the axis of the MRI being horizontal, the beam being inclined with respect to a horizontal plane.

FIG. 5 represents schematically a side view of a nozzle for use in an example embodiment of the present disclosure.

FIG. 6 is a flowchart representing a method according to an example embodiment of the present disclosure.

FIG. 7 is a perspective view of an apparatus according to an example embodiment of the present disclosure.

The drawings of the figures are neither drawn to scale nor proportioned. Generally, identical components are denoted by the same reference numerals in the figures.

### DETAILED DESCRIPTION

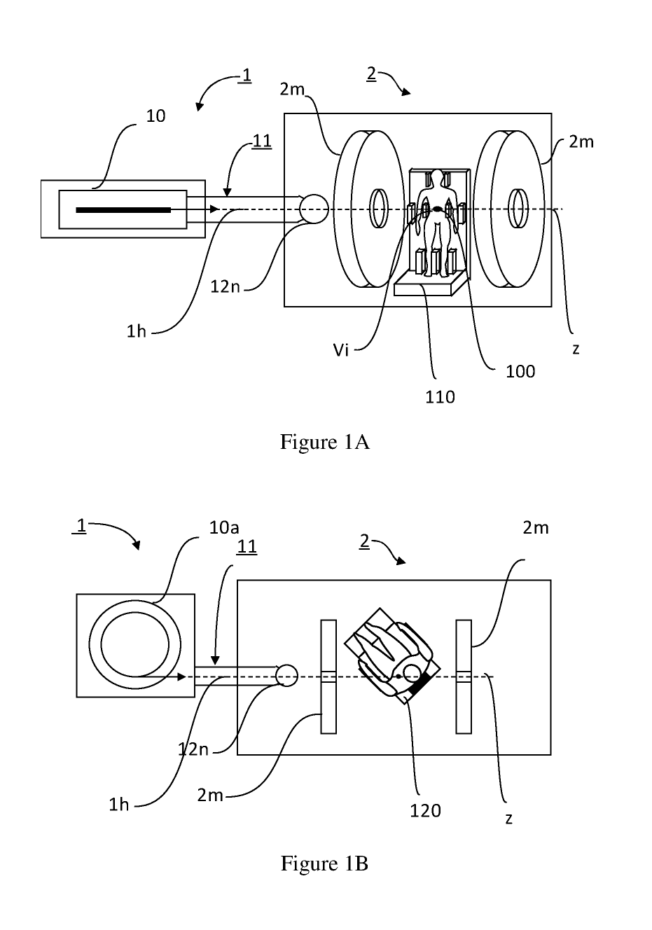

FIG. 1A represents schematically a side view of a medical device according to one embodiment of the present disclosure. The apparatus may comprise a hadron therapy device 1 including hadron source 10. The hadron source 10 may include an accelerator 10a. Suitable accelerators may include, for example, a cyclotron, a synchro-cyclotron, a synchrotron, a laser accelerator, or the like. The energy of the particles of the hadron beam 1h when extracted from the accelerator may be between 60 MeV and 400 MeV, e.g., between 210 MeV and 250 MeV for proton beam, and up to 400 MeV for a carbon beam. A beam transport line 11 may lead the hadron beam 1h from the accelerator to a nozzle 12n. The beam transport line may be under vacuum. The nozzle 12n may perform the functions of shaping and/or directing the beam according to the precise needs of the treatment plan. For example, the nozzle may comprise scanning magnets for directing the beam to a sequence of target spots inside the target volume 40. When no scanning is performed, the beam path may be along a neutral beam path. When scanning is applied, for example, for reaching a spot away from the center 100, the beam path may deviate slightly from the neutral path. The nozzle may also comprise tools for quality assurance, such as device(s) for measuring the energy or intensity of the beam. The nozzle may be prolonged by a beam transport line, e.g., to arrive as near as possible to the patient, such that the path of the beam not under vacuum is minimized. The apparatus may also comprise a magnetic resonance imaging device (MRI) represented schematically by box 2 and comprising a main magnet 2m for producing the main magnetic field B₀ of the MRI along the axis z of the MRI. Other components, not represented, but known in the art, may comprise RF-excitation coils, gradient coils in the three directions X, Y, Z, and antennas. The MRI 2 may be designed for having a region of space wherein the magnetic field B₀ produced by the main magnet 2m meets requirements regarding intensity and direction for allowing acquisition of quality MRI images. This region of space may be the MRI imaging volume Vi.

In the example of FIG. 1A, the path of the hadron beam 1h is represented as collinear with the axis z of the MRI. However, embodiments of the present disclosure may deviate from this collinearity and have a beam path 1h forming an angle, e.g., up to 20°, with the axis z of the MRI. This angular deviation may be obtained, for example, by rotating the MRI 2.

In one embodiment, the hadron therapy device 1 may be located at a distance from the MRI 2. The distance may be, for example, 2 m or larger, measured from the center 100 of the imaging volume Vi to the exit of the nozzle 12m. In embodiments where the hadron therapy apparatus 1 and the MRI 2 are apart, the influence of the stray field of the hadron therapy apparatus on the MRI (and vice versa) may be minimized. Moreover, in the embodiments of FIG. 1, and FIG. 3, where the neutral beam path 1h is parallel to the axis of the MRI, the deviated beam path may deviate only from a reduced angle from the neutral beam path. Therefore, the influence of the main filed B₀ may be reduced with respect to a situation where the distance would be smaller and the deviation angles larger. The source-axis distance (SAD), i.e., the distance between a virtual source located in the scanning magnets and the center 100, is larger, and therefore the strength of the scanning magnets may be reduced, and the scanning magnets may have smaller apertures.

The patient support 110 represented in the example of FIG. 1 is a patient support for a standing patient. The support 110 may comprise a rotation mechanism for rotating the patient around a vertical axis. One of ordinary skill may implement other supports for immobilizing the patient in a position in embodiments of the present disclosure.

FIG. 1B represents schematically a top view of the medical device 1 of FIG. 1A. However, the patient support in FIG. 1B is a seat 120. The seat may be rotatable around a vertical axis, and is represented as making an angle of about 45° with respect to the z axis of the MRI in the example of FIG. 1B.

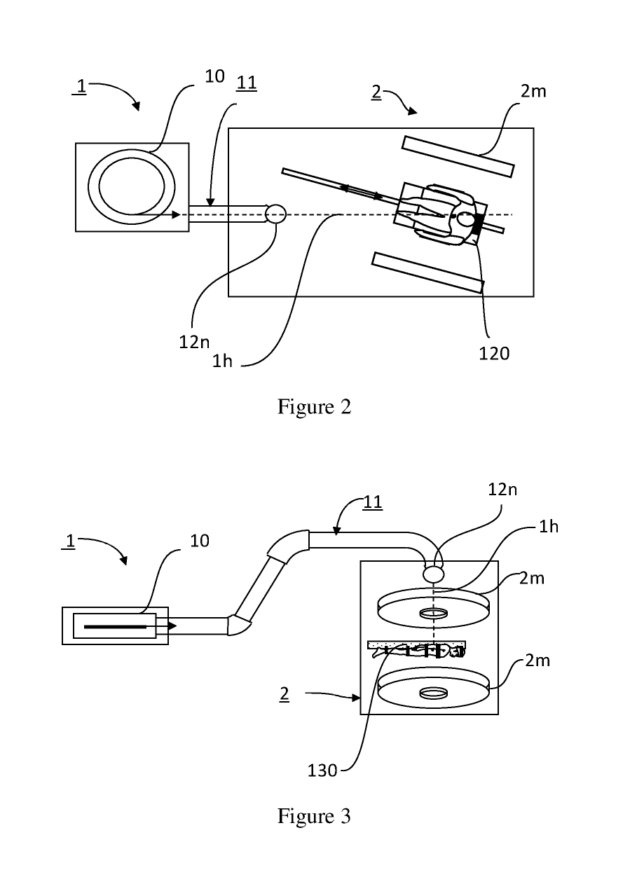

FIG. 2 represents schematically a top view of a medical device 1 wherein the nozzle 12n is positioned for directing a beam in a direction substantially perpendicular to the direction of the axis of the MRI, the axis z of the MRI being horizontal. In the example shown, there is an angle α between the hadron beam and a perpendicular to the axis z of the MRI. This may give more flexibility in planning treatments. The variation of the angle may be obtained, for example, by mounting the MRI 2 on a rotating platform.

The patient support 120 represented in the example of FIG. 2 is a patient support for a seated patient. The support 120 may comprise a rotation mechanism for rotating the patient around a vertical axis and/or a horizontal axis and/or for tilting the patient backwards or forwards. The support may be a chair, as depicted, an ergonomic kneeling chair, or the like. An ergonomic kneeling chair may use less space than is used in the narrow opening of the MRI.

FIG. 3 represents schematically a side view of a medical device according to one embodiment wherein the nozzle is positioned for directing a beam substantially in the direction of the axis z of the MRI, the axis of the MRI being vertical. In the example shown, the beam is led from the hadron source 10 to the nozzle 12n through a beam transport line 11 comprising a number of bending magnets. Alternatively, the hadron source 10 may be located above or below the MRI 2 and may produce a beam in a vertical direction.

The patient support 130 represented in the example of FIG. 3 is a patient support for a patient in prone position. The support 130 may comprise a rotation mechanism for rotating the patient around a horizontal axis.

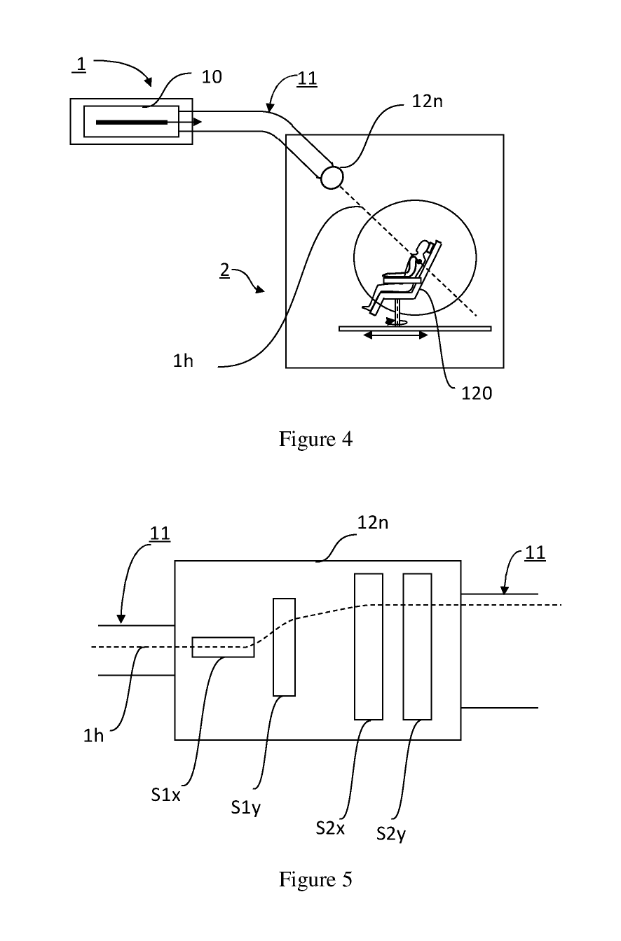

FIG. 4 represents schematically a side view of a medical apparatus according to one embodiment wherein the nozzle is positioned for directing a beam in a direction substantially perpendicular to the direction of the axis of the MRI, the axis of the MRI being horizontal, the beam being inclined with respect to a horizontal plane. The inclination angle may be any value, such as 45° (as shown), or 60°, or 30°, or the like.

FIG. 5 represents schematically a side view of a nozzle for use in embodiments of the present disclosure. This nozzle may be designed for the spot scanning beam delivery, in the parallel scanning mode, as described in Pedroni et al., “The PSI Gantry 2: a second generation proton scanning gantry”, Z. Med. Phys. 14 (2004), pp. 25-34. The nozzle may comprise a first pair of scanning magnets 11x, S1y, for deviating the beam path from the neutral line in the directions x and y, and a second pair of scanning magnets for redirecting the beam parallel to the neutral line. The use of such scanning in the embodiments of FIG. 1 and FIG. 3 may allow the hadron beam 1n to remain parallel to the B₀ field, and therefore little to no deviation of the hadron beam by the B₀ field may occur. The part of the beam transport line at the left of FIG. 5 may transport the beam from the hadron source to the nozzle 12n. In some embodiments of the present disclosure, an additional part of beam transport line 11 may be between the exit of the nozzle and the patient, thereby helping keep the part of the path not under vacuum as short as possible.

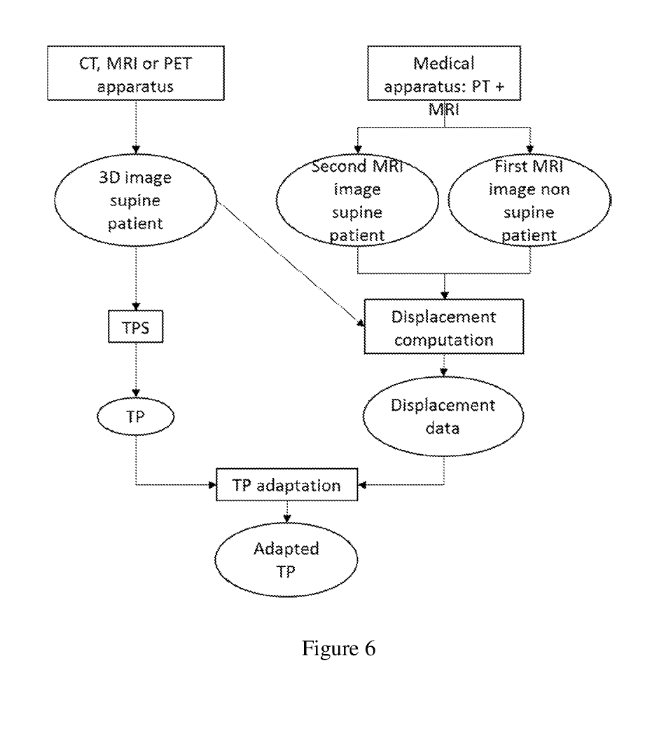

FIG. 6 is a flowchart of a method according to an embodiment of the present disclosure. The rectangular boxes represent apparatuses and/or computers performing operations according to software, and the ovals represent the data flowing between the boxes. The left-hand branch of the diagram represents a traditional establishment of a treatment plan: A CT scanner, MRI, and/or PET imaging device acquires a 3D image of a patient, the patient lying in supine position. For example, the Treatment Planning System (TPS) may be a Raystation (Research), a Pinnacle (Philips), a Xio (Elekta), or others, and may provide a treatment plan TP. The right-hand branch of the diagram represents the operations performed before the treatment, in order to adapt the treatment plan TP to the displacement of organs and the target volume when the patient is in the non-supine position in which the treatment will be performed. The patient being positioned in the medical apparatus (PT+MRI), and positioned in the non-supine position wherein the treatment will be performed, a first MRI image may be acquired.

In one embodiment, this image may be compared with the 3D image acquired in the left-hand part of the diagram, and may be compared by a computer performing a displacement computation for obtaining displacement data representing the displacement of the organs of the patient and of the target volume. These displacement data may be provided to a computer, together with the treatment plan TP, for performing an adaption of the treatment plan and for providing the adapted treatment plan.

In another embodiment, a second MRI image may be acquired, the patient being in supine position, i.e., the position in which the 3D image was acquired. The displacement computer may compute displacement data from the comparison of said first and second MRI images. The second MRI image may be acquired with the MRI of the apparatus and/or in another MRI. These displacement data may be provided to a computer, together with the treatment plan TP, for performing an adaption of the treatment plan and for providing the adapted treatment plan.

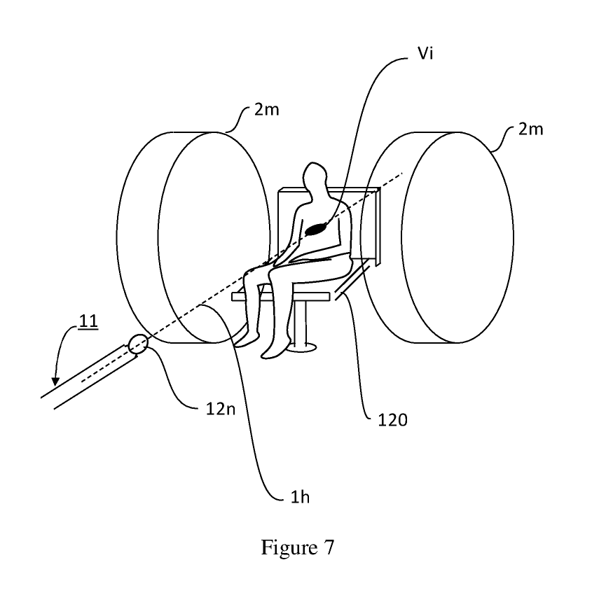

FIG. 7 is a perspective view of an apparatus according to an example embodiment of the present disclosure and having a seated patient support 120. The MRI 2 may be an open MRI. An example of an open MRI is the MRopen apparatus obtainable from Paramed Medical srl and described in U.S. Pat. No. 7,944,208. This apparatus may be modified by providing an aperture or window for allowing the beam to pass through, e.g., along the z axis, for the embodiments of FIG. 1A, FIG. 1B, and FIG. 3.

The MRI used in embodiments of the present disclosure, and in the examples represented in FIGS. 1A to 5 and FIG. 7 may comprise two coils at a distance, providing a space between these two coils. These devices are generally known as “open MRIs.” Such MRIs may allow the patient to not be confined in a narrow bore where he might suffer from claustrophobia. FIGS. 2, 4 and 7 show examples where the beam reaches the patient through this opening. In addition, if the patient is siting, standing or lying in a more open space, this may allow the rotation of the patient support for directing the beam under a choice of angles to the patient.

Using the device and methods of the present disclosure, one may treat patients using a hadron therapy device without a gantry. Accordingly, the cost of the apparatus may be reduced, and the space required for installing the device also reduced. It may also be possible to adapt a treatment plan to movements of organs resulting from the displacement of a patient from a supine position in which treatment plan imaging was performed to a non-supine position for performing the treatment. The availability of the MRI in the hadron therapy apparatus may thus allow treatment in non-supine position, because the treatment plan may be adapted as needed.

1.-14. (canceled)

15. A medical apparatus comprising:
a hadron therapy device comprising a hadron source and a fixed nozzle configured to direct a hadron treatment beam along a beam path to a target volume;
a magnetic resonance imaging device comprising:
two coils arranged at a distance and configured to generate a magnetic field in a direction along a first axis within an imaging volume locating between the two coils and around the first axis,
the magnetic resonance imaging device acquiring magnetic resonance data in the imaging volume,
the imaging volume enclosing the target volume and having a center; and
a patient support system configured to support a patient in a non-supine position,
wherein the nozzle is positioned to direct the hadron treatment beam along a beam axis, the beam axis arranged at a predetermined angle within a range comprising within 20° parallel to the first axis or within 20° perpendicular to the first axis.

16. The medical apparatus of claim 15, wherein the nozzle is positioned to direct the hadron treatment beam substantially along the first axis, and an angle between the direction of the hadron treatment beam and the first axis is less than or equal to 20°.

17. The medical apparatus of claim 15, wherein the nozzle is positioned to direct the hadron treatment beam substantially perpendicular to the first axis, and an angle between the direction of the hadron treatment beam and a second axis perpendicular to the first axis is less than or equal to 20°.

18. The medical apparatus of claim 15, wherein the patient support system is further configured to support the patient in a seated position.

19. The medical apparatus of claim 15, wherein the patient support system is further configured to support the patient in a standing position.

20. The medical apparatus of claim 15, wherein the patient support system is further configured to support the patient in a prone position.

21. The medical apparatus of claim 15, wherein the patient support system is rotatable around a vertical axis.

22. The medical apparatus of claim 15, wherein the patient support system is rotatable around a horizontal axis.

23. The medical apparatus of claim 22, wherein the horizontal axis is a longitudinal axis of the patient support system.

24. The medical apparatus of claim 15, further comprising a controller configured to:
obtain a three-dimensional image of a region of the patient in a supine position, the region enclosing the target volume using at least one of a CT scan, the magnetic resonance imaging device, and a PET imaging system;
determine a treatment plan based on the three-dimensional image;
determine a displacement of the target volume within the patient resulting from the patient being moved from the supine position to a non-supine position;
adapt the treatment plan to the displacement; and
position the patient in the medical apparatus in the non-supine position.

25. The medical apparatus of claim 24, wherein the controller is further configured to:
obtain a first MRI image from the magnetic resonance imaging device with the patient in the non-supine position;
obtain a second MRI image from the magnetic resonance imaging device with the patient being in the supine position; and
determine the displacement of the target volume based on a comparison of the first MRI image and the second MRI image.

26. The medical apparatus of claim 24, wherein the controller is further configured to:
obtain a first MRI image from the magnetic resonance imaging device with the patient in the non-supine position; and
determine the displacement of the target volume based on a comparison of the first MRI image and the three-dimensional image.

27. The medical apparatus of claim 24, wherein the treatment plan includes a plurality of spots each having a spot position in the target volume, and wherein the controller is further configured to:
irradiate one or more spots of the plurality of spots;
acquire an image of the imaging volume using the magnetic resonance imaging device;
compare the image with the three-dimensional image;
adapt the treatment plan based on the comparison; and
repeat the irradiating, acquiring, comparing, and adapting until all spots of the plurality have been irradiated.

28. The medical apparatus of claim 15, further comprising a controller configured to:
obtain a three-dimensional image of the target volume of the patient in a non-supine position using at least one of a CT scan, the magnetic resonance imaging device, and a PET imaging system;
determine a treatment plan based on the three-dimensional image; and
position the patient in the medical apparatus in the non-supine position.

29. The medical apparatus of claim 15, wherein the nozzle is positioned at a distance larger than 2 m from the center.

30. The medical apparatus of claim 15, wherein the nozzle is positioned to direct the hadron treatment beam in a first direction along the beam path toward a first pair of magnets configured to steer the beam in two orthogonal directions perpendicular to the first direction and toward a second pair of magnets configured to steer the beam in a direction parallel to the first direction.

31. A method for preparing a treatment plan for treating a target volume of a patient, comprising:
obtaining a three-dimensional image of a region of the patient in a supine position, the region enclosing the target volume using at least one of a CT scan, a magnetic resonance imaging device, and a PET imaging system;
determining a treatment plan based on the three-dimensional image;
determining a displacement of the target volume within the patient resulting from the patient being moved from the supine position to a non-supine position;
adapting the treatment plan to the displacement; and
positioning the patient in a medical apparatus in the non-supine position.

32. The method of claim 31, further comprising:
obtaining a first MRI image from the magnetic resonance imaging device with the patient in the non-supine position;
obtaining a second MRI image from the magnetic resonance imaging device with the patient being in the supine position; and
determining the displacement of the target volume based on a comparison of the first MRI image and the second MRI image.

33. The method of claim 31, further comprising:
obtain a first MRI image from the magnetic resonance imaging device with the patient in the non-supine position; and
determining the displacement of the target volume based on a comparison of the first MRI image and the three-dimensional image.

34. The method of claim 24, wherein the treatment plan includes a plurality of spots each having a spot position in the target volume, and wherein the method further comprises:
irradiating one or more spots of the plurality of spots;
acquiring an image of an imaging volume of the magnetic resonance imaging device using the magnetic resonance imaging device;
comparing the image with the three-dimensional image;
adapting the treatment plan based on the comparison; and
repeating the irradiating, acquiring, comparing, and adapting until all spots of the plurality have been irradiated.
